SEQUENCE LISTING (24 sequences)

SEQ ID NO: 1 - protein, 955 aa, synthetic construct
  MUTAGEN 10
  MUTAGEN 14
  MUTAGEN 17
  MUTAGEN 256
  MUTAGEN 488
  REGION 724..955: note = human Fc sequence
QSTIEEQAKYFLDHFNAEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 955 aa, synthetic construct
  MUTAGEN 10
  MUTAGEN 14
  MUTAGEN 17
  MUTAGEN 256
  MUTAGEN 488
  REGION 724..955: note = human Fc sequence
QSTIEEQAKYFLDYFNAEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 955 aa, synthetic construct
  MUTAGEN 10
  MUTAGEN 14
  MUTAGEN 17
  MUTAGEN 256
  MUTAGEN 488
  REGION 724..955: note = human Fc sequence
QSTIEEQAKYFLDHFNVEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 955 aa, synthetic construct
  MUTAGEN 10
  MUTAGEN 14
  MUTAGEN 17
  MUTAGEN 256
  MUTAGEN 488
  REGION 724..955: note = human Fc sequence
QSTIEEQAKYFLDYFNVEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 955 aa, synthetic construct
  MUTAGEN 17
  MUTAGEN 256
  MUTAGEN 488
  REGION 724..955: note = human Fc sequence
QSTIEEQAKTFLDKFNAEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 955 aa, synthetic construct
  MUTAGEN 17
  MUTAGEN 256
  MUTAGEN 488
  REGION 724..955: note = human Fc sequence
QSTIEEQAKTFLDKFNVEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 955 aa, synthetic construct
  MUTAGEN 17
  MUTAGEN 256
  MUTAGEN 488
  REGION 724..955: note = human Fc sequence
QSTIEEQAKTFLDKFNPEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 955 aa, synthetic construct
  MUTAGEN 10
  MUTAGEN 256
  MUTAGEN 488
  REGION 724..955: note = human Fc sequence
QSTIEEQAKYFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 955 aa, synthetic construct
  MUTAGEN 10
  MUTAGEN 256
  MUTAGEN 488
  REGION 724..955: note = human Fc sequence
QSTIEEQAKIFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 955 aa, synthetic construct
  MUTAGEN 10
  MUTAGEN 256
  MUTAGEN 488
  REGION 724..955: note = human Fc sequence
QSTIEEQAKMFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 955 aa, synthetic construct
  MUTAGEN 10
  MUTAGEN 256
  MUTAGEN 488
  REGION 724..955: note = human Fc sequence
QSTIEEQAKFFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 955 aa, synthetic construct
  MUTAGEN 14
  MUTAGEN 256
  MUTAGEN 488
  REGION 724..955: note = human Fc sequence
QSTIEEQAKTFLDWFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 955 aa, synthetic construct
  MUTAGEN 25
  MUTAGEN 256
  MUTAGEN 488
  REGION 724..955: note = human Fc sequence
QSTIEEQAKTFLDKFNHEAEDLFYLSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 955 aa, synthetic construct
  MUTAGEN 17
  MUTAGEN 256
  MUTAGEN 488
  REGION 724..955: note = human Fc sequence
QSTIEEQAKTFLDKFNDEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 955 aa, synthetic construct
  MUTAGEN 10
  MUTAGEN 256
  MUTAGEN 488
  REGION 724..955: note = human Fc sequence
QSTIEEQAKQFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 955 aa, synthetic construct
  MUTAGEN 17
  MUTAGEN 256
  MUTAGEN 488
  REGION 724..955: note = human Fc sequence
QSTIEEQAKTFLDKFNEEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 955 aa, synthetic construct
  MUTAGEN 17
  MUTAGEN 256
  MUTAGEN 488
  REGION 724..955: note = human Fc sequence
QSTIEEQAKTFLDKFNGEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 955 aa, synthetic construct
  MUTAGEN 14
  MUTAGEN 256
  MUTAGEN 488
  REGION 724..955: note = human Fc sequence
QSTIEEQAKTFLDHFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 955 aa, synthetic construct
  MUTAGEN 14
  MUTAGEN 256
  MUTAGEN 488
  REGION 724..955: note = human Fc sequence
QSTIEEQAKTFLDQFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 955 aa, synthetic construct
  MUTAGEN 14
  MUTAGEN 256
  MUTAGEN 488
  REGION 724..955: note = human Fc sequence
QSTIEEQAKTFLDYFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 955 aa, synthetic construct
  MUTAGEN 14
  MUTAGEN 256
  MUTAGEN 488
  REGION 724..955: note = human Fc sequence
QSTIEEQAKTFLDMFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 955 aa, synthetic construct
  MUTAGEN 256
  MUTAGEN 488
  REGION 724..955: note = human Fc sequence
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 805 aa, Homo sapiens [sequence omitted]

(19) United States
(12) Patent Application Publication
(10) Pub. No.: US 2025/0327052 A1
(43) Pub. Date: Oct. 23, 2025

(54) Recombinant/Fusion Polypeptides Comprising Mutated Angiotensin Converting Enzyme 2 (ACE2)

(71) Applicant: Agency for Science, Technology and Research, Singapore (SG)

(72) Inventors: Cheng-I Wang, Singapore (SG); Chia Yin Lee, Singapore (SG); Rabiatul Adawiyah Binte Minhat, Singapore (SG); Bei Wang, Singapore (SG); Roland Huber, Singapore (SG); Louis DeFalco, Singapore (SG); Ching-Wen Huang, Singapore (SG)

(21) Appl. No.: 18/706,143

(22) PCT Filed: Oct. 27, 2022
(86) PCT No.: PCT/SG2022/050775

§ 371 Date: Apr. 30, 2024

(30) Foreign Application Priority Data
Nov. 1, 2021 (SG) 10202112143T

(51) Int. Cl. C12N 9/48; A61P 31/14

(52) U.S. Cl. CPC C12N 9/485; A61P 31/14; C12Y 304/17023; C07K 2319/30; C07K 2319/31

(57) ABSTRACT

The invention relates to recombinant mutated angiotensin converting enzyme 2 (ACE2) comprising one or more amino acid substitutions at amino acid positions T27, F28, K31, H34, Y41 and Q42 which have improved binding affinity to SARS-CoV-1 and/or SARS-CoV-2. It also relates to combinatorial mutant ACE2 comprising T27Y/K31 H/H34A, T27YZ K31 H/H134V and T27Y/K31 Y/H134V and the use of said mutant ACE2 for treating and preventing coronavirus infection.

### RELATED APPLICATION

This application is the U.S. National Stage of International Application No. PCT/SG2022/050775, filed Oct. 27, 2022, which designates the U.S., published in English, and claims priority under 35 U.S.C. § 119 or 365(c) to Singapore Application No. 10202112143T, filed Nov. 1, 2021. The entire teachings of the above applications are incorporated herein by reference.

### TECHNICAL FIELD

The present disclosure relates broadly to recombinant/fusion polypeptides comprising mutated angiotensin converting enzyme 2 (ACE2) which are capable of binding to viruses, such as SARS-CoV-1 and SARS-CoV-2. Also provided are compositions comprising the polypeptides, a method of treatment employing the polypeptides, and other uses thereof.

### BACKGROUND

Although neutralizing antibodies, including those elicited by vaccines, against a particular virus can effectively block infection, they are often too specific to inhibit viral variants that arise from natural mutations during a pandemic. Also, neutralizing antibodies raised against one virus often do not cross-react with other viruses that utilize the same entry receptor. For example, the vast majority of anti-SARS-CoV-1 (SARS) antibodies do not react SARS-CoV-2 (COVID-19) and vice versa.

A number of coronaviruses, including SARS-CoV-1 and SARS-CoV-2, use host cell angiotensin converting enzyme 2 (ACE2) as the entry receptor to initiate infection. Hence, the extracellular domain of the ACE2, may potentially serve as a decoy for all ACE2-dependent viruses by competing with host cell ACE2 for binding to the spike receptor binding domains of coronaviruses.

However, native (wildtype) ACE2 often has low affinity for coronavirus spike proteins. Accordingly, there is a need to improve the affinity of ACE2 against multiple coronaviruses and their variants in order for ACE2 to be suitable for use as a therapeutic.

### SUMMARY

In one aspect, there is provided a recombinant/fusion polypeptide comprising a mutated angiotensin converting enzyme 2 (ACE2) protein, or fragment thereof, and an immunoglobulin fragment.

In one embodiment, the recombinant/fusion polypeptide comprises an immunoglobulin Fc fragment (region/domain), a protein capable of extending the half-life of the recombinant/fusion polypeptide (such as albumin, human albumin, and the like), linkers capable of enhancing binding valency (for example a rigid linker or a flexible linker, such as a GGGGS repeat), or combinations thereof, for example wherein the immunoglobulin Fc fragment is an IgG Fc fragment, such as a human IgG Fc fragment.

In one embodiment, the immunoglobulin fragment comprises the sequence:

(SEQ ID NO: 23)
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

In one embodiment, the recombinant/fusion polypeptide binds to a viral protein.

In one embodiment, the viral protein is a Coronavirus protein or a Flaviviridae protein.

In one embodiment, the coronavirus is a Severe Acute Respiratory Syndrome virus, such as severe acute respiratory syndrome 1 (SARS-CoV-1), severe acute respiratory syndrome 2 (SARS-CoV-2), and the like.

In one embodiment, the ACE2 protein or fragment thereof comprises one or more mutations, two or more mutations, three or more mutations, four or more mutations, five or more mutations, or six or more mutations, or seven or more mutations, or eight or more mutations.

In one embodiment, the ACE2 protein or fragment thereof comprises one or more mutations at amino acid positions selected from the group comprising 27, 28, 31, 34, 41, and 42, for example positions 27, 28, 31, 34, 41 and 42 as set forth in SEQ ID NO: 24.

In one embodiment, the ACE2 protein or fragment thereof comprises mutations at amino acid positions selected from the group comprising 27, 31 and 34, for example positions 27, 31 and 34 as set forth in SEQ ID NO: 24.

In one embodiment, the ACE2 protein or fragment thereof comprises one or more mutations selected from the group consisting of:
T27A, T27R, T27N, T27D, T27E, T27Q, T27G, T27H, T271, T27L, T27K, T27M, T27F, T27P, T27S, T27T, T27W, T27Y, T27V;
Q42A, Q42R, Q42N, Q42D, Q42E, Q42Q, Q42G, Q42H, Q421, Q42L, Q42K, Q42M, Q42F, Q42P, Q42S, Q42T, Q42W, Q42Y, Q42V;
H34A, H34R, H34N, H34D, H34E, H34Q, H34G, H34H, H341, H34L, H34K, H34M, H34F, H34P, H34S, H34T, H34W, H34Y, H34V;
K31A, K31R, K31N, K31D, K31E, K31Q, K31G, K31H, K311, K31L, K31K, K31M, K31F, K31P, K31S, K31T, K31W, K31Y, K31V;
F28A, F28R, F28N, F28D, F28E, F28Q, F28G, F28H, F281, F28L, F28K, F28M, F28F, F28P, F28S, F28T, F28W, F28Y, F28V;
Y41A, Y41R, Y41N, Y41D, Y41E, Y41Q, Y41G, Y41H, Y411, Y41L, Y41K, Y41M, Y41F, Y41P, Y41S, Y41T, Y41W, Y41Y, and Y41V.

In one embodiment, the ACE2 protein or fragment thereof comprises one or more mutations selected from the group consisting of:
T27A, T27R, T27N, T27D, T27E, T27Q, T27G, T27H, T271, T27L, T27K, T27M, T27F, T27P, T27S, T27W, T27Y, T27V;
Q42A, Q42R, Q42N, Q42D, Q42E, Q42H, Q421, Q42L, Q42K, Q42M, Q42F, Q42P, Q42S, Q42T, Q42W, Q42Y, Q42V;
H34A, H34R, H34N, H34D, H34E, H34Q, H34G, H341, H34L, H34M, H34F, H34P, H34S, H34T, H34W, H34Y, H34V;
K31A, K31R, K31N, K31Q, K31H, K311, K31K, K31M, K31F, K31W, K31Y, K31V;
F28W, F28Y;
Y41H, and Y41F.

In one embodiment, the ACE2 protein or fragment thereof comprises one or more mutations selected from the group consisting of: T27Y, K31Y, K31H, H34A, H34V, and combinations thereof.

In one embodiment, the ACE2 protein or fragment thereof comprises one of the following combination of mutations:
T27Y/K31H/H34A,
T27Y/K31Y/H34A,
T27Y/K31H/H34V, or
T27Y/K31Y/H34V.

In one embodiment, the ACE2 protein or fragment thereof comprises the following combination of mutations: T27Y/K31H/H34A.

In one embodiment, the recombinant/fusion polypeptide is capable of binding to a virus, optionally the recombinant/fusion polypeptide is capable of binding blocking/interfering/inhibiting/hindering/disrupting the binding of a virus (such as SARS-CoV-2) binding to a cell entry receptor.

In one embodiment, the recombinant/fusion polypeptide is capable of neutralising or mediating (or initiating) the neutralisation of a virus, such as SARS-CoV-2.

In one embodiment, the ACE2 protein or fragment thereof comprises one or more mutations that abolishes the natural peptidase activity of ACE2.

In one embodiment, the ACE2 protein or fragment thereof comprises a mutation at amino acid position 273 and/or 505, for example positions 273 and/or 505 as set forth in SEQ ID NO: 24.

In one embodiment, the ACE2 protein or fragment thereof further comprises a R273Q and/or a H505L mutation, such as both R273Q and H505L.

In one embodiment, the recombinant/fusion protein or fragment thereof comprising a R273Q and/or H505L mutation comprises the sequence:

(SEQ ID NO: 22)
QSTIEEQAKTFLDKFNHEAEDLFYQSSLASWNYNTNITEENVQNMNNAG
DKWSAFLKEQSTLAQMYPLQEIQNLTVKLQLQALQQNGSSVLSEDKSKR
LNTILNTMSTIYSTGKVCNPDNPQECLLLEPGLNEIMANSLDYNERLWA
WESWRSEVGKQLRPLYEEYVVLKNEMARANHYEDYGDYWRGDYEVNGVD
GYDYSRGQLIEDVEHTFEEIKPLYEHLHAYVRAKLMNAYPSYISPIGCL
PAHLLGDMWGQFWTNLYSLTVPFGQKPNIDVTDAMVDQAWDAQRIFKEA
EKFFVSVGLPNMTQGFWENSMLTDPGNVQKAVCHPTAWDLGKGDFRILM
CTKVTMDDFLTAHHEMGHIQYDMAYAAQPFLLRNGANEGFHEAVGEIMS
LSAATPKHLKSIGLLSPDFQEDNETEINFLLKQALTIVGTLPFTYMLEK
WRWMVFKGEIPKDQWMKKWWEMKREIVGVVEPVPHDETYCDPASLFLVS
NDYSFIRYYTRTLYQFQFQEALCQAAKHEGPLHKCDISNSTEAGQKLFN
MLRLGKSEPWTLALENVVGAKNMNVRPLLNYFEPLFTWLKDQNKNSFVG
WSTDWSPYADQSIKVRISLKSALGDKAYEWNDNEMYLFRSSVAYAMRQY
FLKVKNQMILFGEEDVRVANLKPRISFNFFVTAPKNVSDIIPRTEVEKA
IRMSRSRINDAFRLNDNSLEFLGIQPTLGPPNQPPVSEPKSCDKTHTCP
PCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVF
SCSVMHEALHNHYTQKSLSLSPGK.

In one embodiment, the recombinant/fusion protein or fragment thereof comprises an amino acid sequence selected from the group consisting of SEQ ID NOS: 1 to 21.

In one embodiment, the recombinant/fusion protein or fragment thereof comprises an amino acid sequence selected from the group consisting of SEQ ID NOs: 1 to 4, for example SEQ ID NO: 1.

In one aspect, there is provided a polynucleotide encoding a recombinant/fusion polypeptide as defined above.

In one aspect, there is provided a vector comprising a polynucleotide as defined above.

In one aspect, there is provided a host cell transfected with or comprising a vector as defined above.

In one aspect, there is provided a composition comprising the recombinant/fusion polypeptide as defined above.

In one aspect, there is provided a recombinant/fusion polypeptide, or a composition as defined above for use as a therapy or drug.

In one aspect, there is provided a recombinant/fusion polypeptide, or a composition as defined above for use for use in treating and/or preventing a viral infection in a subject.

In one aspect, there is provided a use of the recombinant/fusion polypeptide of or a composition as defined above in the manufacture of a medicament for treating and/or preventing a viral infection in a subject.

In one aspect, there is provided a method of treating and/or preventing a viral infection in a subject in need thereof, comprising administering the recombinant/fusion polypeptide or a composition as defined above to the subject.

In one embodiment, the viral infection is caused by a virus from the coronaviridae family, such as coronavirus, in particular severe acute respiratory syndrome 1 (SARS-CoV-1), severe acute respiratory syndrome 2 (SARS-CoV-2), or the like.

### Definitions

The term “recombinant polypeptide” as used herein refers to a polypeptide that has been made using any recombinant DNA technique.

The term “fusion polypeptide” as used herein refers to a polypeptide comprising at least two domains that have been encoded by separate genes and joined such that the two domains are transcribed and translated as a single unit, thereby producing a single polypeptide.

The term “polypeptide” as used herein refers a single linear chain of any number of amino acid residues connected via peptide bonds.

The term “angiotensin converting enzyme 2” or “ACE2” as used herein refers a protein that belongs to the angiotensin-converting enzyme family of dipeptidyl carboxydipeptidases and has considerable homology to human angiotensin 1 converting enzyme. ACE2 catalyzes the cleavage of angiotensin I into angiotensin 1-9, and angiotensin II into the vasodilator angiotensin 1-7. ACE2 is known to be expressed in various human organs, and its organ- and cell-specific expression suggests that it may play a role in the regulation of cardiovascular and renal function, as well as fertility. In addition, the encoded protein is a functional receptor for the spike glycoprotein of the human coronavirus HCoV-NL63 and the human severe acute respiratory syndrome coronaviruses, SARS-CoV and SARS-CoV-2, the latter is the causative agent of coronavirus disease-2019 (COVID-19). Multiple splice variants have been found for this gene and the dACE2 (or MIRb-ACE2) splice variant has been found to be interferon inducible. Unless specified, the term ACE2 generally refers to human ACE2 (Uniprot ID Q9BYF1 and/or NCBI reference sequence NP_068576.1) or as encoded by nucleotide NM 021804.3.

The term “Immunoglobulin” or “antibody” as used herein refers to a bivalent Y-shaped molecule comprising two identical heavy chains and two identical light chains. Disulfide bonds link together the heavy and light chain pairs as well as the two heavy chains. Each chain consists of one variable domain that varies in sequence and is responsible for antigen binding, these are known as the V_{H} and V_{L} domains for the heavy and light chains respectively. Each chain also consists of at least one constant domain. In the light chain there is a single constant domain (C_{L}) and in the heavy chain there are at least three (C_{H}1, C_{H}2 and C_{H}3), sometimes four (C_{H}4) depending on the isotype. In humans there are five different classes or isotypes of antibodies including IgA (which includes IgA1 and IgA2), IgD, IgE, IgG (which includes subclasses IgG1, IgG2, IgG3 and IgG4) and IgM.

The term “immunoglobulin fragment” as used herein refers to an antibody that may be, but are not limited to Fab, modified Fab, Fab′, modified Fab′, F(ab′)2, Fv, Fab-Fv, Fab-dsFv, single domain antibodies (e.g. VH or VL or VHH), scFv, bi, tri or tetra-valent antibodies, Bis-scFv, diabodies, triabodies, tetrabodies and epitope-binding fragments of any of the above (see for example Holliger and Hudson, 2005, Nature Biotech. 23 (9): 1126-1136; Adair and Lawson, 2005, Drug Design Reviews-Online 2 (3), 209-217). The methods for creating and manufacturing these antibody fragments are well known in the art (see for example Verma et al., 1998, Journal of Immunological Methods, 216, 165-181).

The term “viral protein” as used herein refers to any protein generated by a virus, for example a viral envelope or a viral capsid protein.

The term “coronavirus” as used herein refers to a group of viruses that are enveloped with a positive-sense single stranded RNA genome and a nucleocapsid or helical symmetry. Coronaviruses are characterised by club-shaped spikes that project from their surface, which in electron micrographs resembles a stellar corona, hence their name. Examples of coronaviruses include but are not limited to SARS-CoV-1, SARS-CoV-2, alphacoronavirus 1, human coronavirus 229E (HCoV-229E), human coronavirus NL63 (HCoV-NL63), human coronavirus OC43 (HCoV-OC43), human coronavirus HKU1 (HCoV-HKU1), betacoronavirus 1, Middle East respiratory syndrome-related coronavirus (MERS-Cov), murine coronavirus, avain coronavirus, porcine coronavirus HKU15, etc.

The term “flaviviridae” as used herein refers to a family of positive, single stranded, enveloped RNA viruses that are mainly spread through arthropod vextors, such as ticks and mosquitoes. Members of the Flaviviridae family include but are not limited to west nile virus, Dengue virus, tick-borne encephalitis virus, yellow fever virus, Zika virus etc.

The term “severe acute respiratory syndrome coronavirus 1” or “SARS-CoV-1” as used herein refers to the strain of coronavirus responsible for severe acute respiratory syndrome (SARS).

The term “severe acute respiratory syndrome coronavirus 2” or “SARS-CoV-2” as used herein refers to the coronavirus responsible for novel coronavirus disease 2019 (COVID-19).

Both SARS-CoV-1 and SARS-CoV-2 are enveloped, positive-sense single stranded RNA viruses that infect the epithelial cells within the lungs. The viruses enter host cells by binding to ACE2 and are known to infect various organisms, including but not limited to humans, bats and cats.

The term “large spike glycoprotein” used interchangeably with “spike protein” and “surface glycoprotein”, abbreviated as “S protein” as used herein refers to the spike protein of SARS-CoV-2. The S protein is a 180-200 kDa trimeric class I fusion protein that consists of two subunits, called the S1 and S2 subunits. The S1 subunit (symbol: CoV_S1, Pfam: PF01600, InterPro: IPR002551) is composed of 672 amino acids (residues 14-685 of the S protein) and is responsible for mediating binding to host cells via interactions with the human receptor angiotensin converting enzyme 2 (ACE2). The S2 (symbol: CoV_S2, Pfam: PF01601, InterPro: IPR002552) subunit is composed of 588 amino acids (residues 686-1273) and mediates viral cell membrane fusion by forming a six-helical bundle via the two-heptad repeat domain. The S protein therefore plays an important role in host cell binding and entry. The S protein amino acid sequence is given in GenBank no. QHD43416.1.

The term “mutation” as used herein refers to a change in the amino acid sequence of a peptide, polypeptide or protein or a change in the nucleic acid sequence encoding the peptide, polypeptide or protein. The four main causes of mutations are spontaneous mutations, mutations due to errors during replication, mutations introduced during DNA repair, and induced mutations caused by mutagens. Mutations can be further classified as large-scale mutations, such as gene duplications, polyploidy, and deletions of large chromosomal regions; and small-scale mutations which typically affect one or a few nucleotides, such as insertions, deletions and substitutions.

The term “neutralisation” as used herein refers to a reduction in viral infectivity, typically by binding the surface of viral particles, such binding viral receptors, thereby blocking a step in the viral replication cycle that precedes viral transcription or synthesis.

The term “subject” as used herein includes patients and non-patients. The term “patient” refers to individuals suffering or are likely to suffer from a medical condition such as a flavivirus infection, while “non-patients” refer to individuals not suffering and are likely to not suffer from the medical condition. “Non-patients” include healthy individuals, non-diseased individuals and/or an individual free from the medical condition. The term “subject” includes humans and animals. Animals include murine and the like. “Murine” refers to any mammal from the family Muridae, such as mouse, rat, and the like.

The term “treatment”, “treat” and “therapy”, and synonyms thereof as used herein refer to both therapeutic treatment and prophylactic or preventative measures, wherein the object is to prevent or slow down (lessen) a medical condition, which includes but is not limited to diseases (such as flavivirus infections), symptoms and disorders. A medical condition also includes a body's response to a disease or disorder, e.g. inflammation. Those in need of such treatment include those already with a medical condition as well as those prone to getting the medical condition or those in whom a medical condition is to be prevented.

The term “therapeutic agent’ as used herein refers to a drug, protein, peptide, gene, chemical compound or other pharmaceutically active ingredient.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

“At least 95% identical” as employed herein is intended to refer to an amino acid sequence which over its full length is 95% identical or more to a reference sequence, such as 96, 97, 98 or 99% identical. Software programmes can be employed to calculate percentage identity.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

In one aspect, there is provided a recombinant/fusion polypeptide comprising a mutated angiotensin converting enzyme 2 (ACE2) protein, or fragment thereof, and an immunoglobulin fragment.

Advantageously, the presently disclosed polypeptides have improved binding affinity to the spike proteins of viruses, such as the receptor binding domain (RBD) of SARS-CoV-1 and SARS-CoV-2. In addition, the disclosed polypeptides have enhanced neutralizing potency against the SARS-CoV-1, SARS-CoV-2, and SARS-CoV-2 variant pseudoviruses. Thus, the presently disclosed polypeptides have a strong potential for use in therapy, for example for the treatment of viral infections such as SARS and COVID-19.

In one embodiment, the recombinant/fusion polypeptide comprises an immunoglobulin Fc fragment (region/domain), a protein capable of extending the half-life of the recombinant/fusion polypeptide (such as albumin, human albumin, and the like), linkers capable of enhancing binding valency (for example a rigid linker or a flexible linker, such as a GGGGS repeat), or combinations thereof, for example wherein the immunoglobulin Fc fragment is an IgG Fc fragment, such as a human IgG Fc fragment.

In various embodiments, the immunoglobulin fragment may include but is not limited to a Fc region/domain, one or more CH regions/domains, a Fab, a Fab′, a F(ab′)2, a single chain Fv (ScFv) and/or Fv fragments, hinge regions/domain, as well as fragments or portions thereof.

In some embodiments, a recombinant/fusion polypeptide may also contain portions of immunoglobulin molecules or antibodies, such as including, but not limited to, all or portions of a constant heavy chain, a variable heavy chain, a constant light chain, a variable light chain, a hinge region, and/or an Fc domain of a Ig, as well as variants thereof. For example, a recombinant/fusion polypeptide as described herein may be combined with portions of a human IgG. In various embodiments, the type of immunoglobulin may include one or more type such as, but is not limited to, IgG, IgE, IgM, IgD, IgA, and IgY. In various embodiments, the type of immunoglobulin may be an immunoglobulin of class IgG1, IgG2, IgG3, IgG4, IgA1, IgA2, or subclass thereof.

In one embodiment, the immunoglobulin fragment is an IgG Fc fragment. In one embodiment, the immunoglobulin fragment is a human IgG Fc fragment, for example a Fc fragment comprising SEQ ID NO: 23. Hence, in one embodiment, the immunoglobulin fragment comprises the sequence:

(SEQ ID NO: 23)
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

In one embodiment, the recombinant/fusion polypeptide binds to a viral protein.

In one embodiment, the viral protein is a Coronavirus protein or a Flaviviridae protein.

In one embodiment, the coronavirus is a Severe Acute Respiratory Syndrome virus, such as severe acute respiratory syndrome 1 (SARS-CoV-1), severe acute respiratory syndrome 2 (SARS-CoV-2), and the like. Thus, in one embodiment, the recombinant/fusion polypeptide binds to SARS-CoV-1 and/or SARS-CoV-2.

In various embodiments, SARS-CoV-2 includes various variants known in the art, such as Alpha variant, Beta variant, Gamma variant, Epsilon variant, Eta variant, lota variant, Kappa variant, Kappa variant, Mu variant, Zeta variant, Delta variant, and the like. In various embodiments, the virus as described herein includes variants such as, but is not limited to, SARS-CoV-2, CoV-2 V483A, CoV-2 G476S, CoV-2 L4551/F456V, CoV-2 V4831, CoV-2 N439K, CoV-2 S494P, CoV-2 D614G, CoV-2 S477N, CoV-2 A222V, CoV-2 Y453F, CoV-2 N501Y, CoV-2 E406W, CoV-2 Q493F, CoV-2 Q493K, CoV-2 L455F, CoV-2 E484K, CoV-2 G485D, CoV-2 F486K, CoV-2 F486V, CoV-2 F486A, CoV-2 F486L, CoV-2 K417N, CoV-2 N501Y/D614G, CoV-2 E406W/D614G, CoV-2 K417N/E484K/N501Y, Alpha (B.1.1.7), Beta (B.1.351), Gamma (P.1), Kappa (B.1.617.1), Delta (B.1.617.2), Epsilon (B.1.429), SARS-CoV-1, and the like.

In various embodiments, the ACE2 protein or fragment thereof may comprise one mutation, or two mutations, or three mutations, or four mutations, or five mutations, or six mutations, or seven mutations, or eight mutations. In various embodiments, the ACE2 protein or fragment thereof may comprise at least one mutation, or at least two mutations, or at least three mutations, or at least four mutations, or at least five mutations, or at least six mutations, or at least seven mutations, or at least eight mutations. In various embodiments, the ACE2 protein or fragment thereof may comprise no more than one mutation, or no more than two mutations, or no more than three mutations, or no more than four mutations, or no more than five mutations, or no more than six mutations, or no more than seven mutations, or no more than eight mutations.

In various embodiments, the mutation may include, but is not limited to substitutions, insertion, deletion, duplication, frameshift, and the like.

In one embodiment, the mutation is a substitution.

In various embodiments, the substitution replaces the amino acids with natural amino acids, such as, but is not limited to, alanine, arginine, asparagine, aspartate, cysteine, glutamine, glutamate, glycine, histidine, isoleucine, leucine, lysine, methionine, phenylalanine, proline, serine, threonine, tryptophan, tyrosine, and valine. In various embodiments, the substitution does not include replacement with cysteine. Thus, in various embodiments, the substitution replaces the amino acids with natural amino acids, such as, but is not limited to, alanine, arginine, asparagine, aspartate, glutamine, glutamate, glycine, histidine, isoleucine, leucine, lysine, methionine, phenylalanine, proline, serine, threonine, tryptophan, tyrosine, and valine.

In various embodiments, the substitution may be a conservative substitution that substitute one for another of similar properties. For example, substitution of one amino acid with another from the same group. In various embodiments, the substitution may include substitution with an amino acid with different properties. In various embodiments, the substitution is made without affecting the biological activity of the recombinant polypeptide as described herein. It would be understood that the mutation as described herein would provide a mutated ACE2 protein having substantially the same structure as a native ACE2 protein. Thus, the mutated ACE2 protein as described herein would be capable of binding to a viral protein or fragments thereof. In various embodiments, the mutated ACE2 protein would have abolished or reduced natural peptidase activity whilst still maintaining its ability to bind to a viral protein or fragments thereof. In various embodiments, the substitution increases binding affinity with a viral protein.

In various embodiments, the mutations may be in a compact region of ACE2 that binds to SARS-CoV and/or SARS-CoV-2 spike glycoproteins. For example, the mutations may be in a location as shown in PDB structures 2AJF (SARS-CoV, ACE2 Uniprot id Q9BYF1; Spike GP Uniprot ID P59594, DOI 10.2210/pdb2AJF/pdb) and 6M0J (SARS-CoV-2, ACE2 Uniprot ID Q9BYF1, Spike GP Uniprot ID P0DTC2; DOI 10.2210/pdb6M0J/pdb).

In various embodiments, the specific positions may be selected to enhance binding stability between ACE2 and SARS-CoV-2 spike glycoprotein. In various embodiments, the specific embodiments may be determined using free energy calculations. Without wishing to be bound by theory, mutations in the present disclosure enhance the complementarity of interfaces by increasing hydrophobicity and/or interdigitation within the contact area while minimizing clashes between residues.

In various embodiments, the ACE2 as used herein refers to the extracellular domain of ACE2. In various embodiments, the ACE2 as used herein refers to amino acid 18 to 740 of human ACE2 (Uniprot ID Q9BYF1 and/or NCBI reference sequence NP_068576.1) or as encoded by nucleotide NM 021804.3. Thus, in one embodiment, the recombinant/fusion polypeptide comprises a mutated extracellular domain of ACE2.

In various embodiments, the recombinant/fusion polypeptide may/may not comprise C-terminal domain of ACE2. In various embodiments, the recombinant/fusion polypeptide may comprise the domain interacting with SARS-CoV-1 and/or SARS-CoV-2. In various embodiments, the recombinant/fusion polypeptide may comprise the domain that interacts with SARS-CoV-1 and/or SARS-CoV-2 spike glycoprotein. In various embodiments, the recombinant/fusion polypeptide may comprise a truncated ACE2 that is capable of binding to SARS-CoV-1 and/or SARS-CoV-2 (such as SARS-CoV-1 and/or SARS-CoV-2 spike glycoprotein). In various embodiments, the recombinant/fusion polypeptide do not comprise truncated ACE2 that does not have binding affinity to SARS-CoV-1 and/or SARS-CoV-2 (such as SARS-CoV-1 and/or SARS-CoV-2 spike glycoprotein).

In one embodiment, the ACE2 protein or fragment thereof comprises one or more mutations at amino acid positions selected from the group comprising 27, 28, 31, 34, 41, and 42. Advantageously, the present inventors have determined that these particular amino acid positions may lead to altered interaction of ACE2 with viral spike proteins, for example may lead to an effect on interface stability between ACE2 and viral spike proteins. Hence, introducing a mutation at one or more of these positions has a higher probability of enhancing ACE2 binding affinity for viral spike proteins.

In one embodiment, the ACE2 protein or fragment thereof comprises mutations at amino acid positions selected from the group comprising 27, 31, 34 and 42. In another embodiment, the ACE2 protein or fragment thereof comprises mutations at amino acid positions selected from the group comprising 27, 31 and 34. In one embodiment, the ACE2 protein or fragment thereof comprises mutations at amino acid positions 27, 31 and 34. Surprisingly, introducing mutations at these particular amino acid positions resulted in a slower dissociation rate against viral spike proteins.

In some embodiments, amino acid positions as recited are calculated from position 1 with reference to ACE2 in accordance to human ACE2 (Uniprot ID Q9BYF1 and/or NCBI reference sequence NP_068576.1) or as encoded by nucleotide NM 021804.3.

In various embodiments, the human ACE2 comprises the sequence:

(SEQ ID NO: 24)
MSSSSWLLLSLVAVTAAQSTIEEQAKTFLDKFNHEAEDLFYQSSLASWN
YNTNITEENVQNMNNAGDKWSAFLKEQSTLAQMYPLQEIQNLTVKLQLQ
ALQQNGSSVLSEDKSKRLNTILNTMSTIYSTGKVCNPDNPQECLLLEPG
LNEIMANSLDYNERLWAWESWRSEVGKQLRPLYEEYVVLKNEMARANHY
EDYGDYWRGDYEVNGVDGYDYSRGQLIEDVEHTFEEIKPLYEHLHAYVR
AKLMNAYPSYISPIGCLPAHLLGDMWGRFWTNLYSLTVPFGQKPNIDVT
DAMVDQAWDAQRIFKEAEKFFVSVGLPNMTQGFWENSMLTDPGNVQKAV
CHPTAWDLGKGDFRILMCTKVTMDDFLTAHHEMGHIQYDMAYAAQPFLL
RNGANEGFHEAVGEIMSLSAATPKHLKSIGLLSPDFQEDNETEINFLLK
QALTIVGTLPFTYMLEKWRWMVFKGEIPKDQWMKKWWEMKREIVGVVEP
VPHDETYCDPASLFHVSNDYSFIRYYTRTLYQFQFQEALCQAAKHEGPL
HKCDISNSTEAGQKLFNMLRLGKSEPWTLALENVVGAKNMNVRPLLNYF
EPLFTWLKDQNKNSFVGWSTDWSPYADQSIKVRISLKSALGDKAYEWND
NEMYLFRSSVAYAMRQYFLKVKNQMILFGEEDVRVANLKPRISFNFFVT
APKNVSDIIPRTEVEKAIRMSRSRINDAFRLNDNSLEFLGIQPTLGPPN
QPPVSIWLIVFGVVMGVIVVGIVILIFTGIRDRKKKNKARSGENPYASI
DISKGENNPGFQNTDDVQTSF.

Thus, in one embodiment a mutation at amino acid positions 27, 28, 31, 34, 41, and 42 refers to a mutation at amino acid positions 27, 28, 31, 34, 41, and 42 as set forth in SEQ ID NO: 24. Accordingly, in one embodiment, the ACE2 protein or fragment thereof comprises one or more mutations at amino acid positions selected from the group comprising 27, 28, 31, 34, 41, and 42 as set forth in SEQ ID NO: 24. In one embodiment, the ACE2 protein or fragment thereof comprises one or more mutations at amino acid positions selected from the group comprising 27, 31, 34 and 42 as set forth in SEQ ID NO: 24. In one embodiment, the ACE2 protein or fragment thereof comprises one or more mutations at amino acid positions selected from the group comprising 27, 31 and 34 as set forth in SEQ ID NO: 24. In one embodiment, the ACE2 protein or fragment thereof comprises mutations at amino acid positions 27, 31 and 34 as set forth in SEQ ID NO: 24.

In one embodiment, the ACE2 protein or fragment thereof comprises one or more mutations selected from the group consisting of:
T27A, T27R, T27N, T27D, T27E, T27Q, T27G, T27H, T271, T27L, T27K, T27M, T27F, T27P, T27S, T27T, T27W, T27Y, T27V;
Q42A, Q42R, Q42N, Q42D, Q42E, Q42Q, Q42G, Q42H, Q421, Q42L, Q42K, Q42M, Q42F, Q42P, Q42S, Q42T, Q42W, Q42Y, Q42V;
H34A, H34R, H34N, H34D, H34E, H34Q, H34G, H34H, H341, H34L, H34K, H34M, H34F, H34P, H34S, H34T, H34W, H34Y, H34V;
K31A, K31R, K31N, K31D, K31E, K31Q, K31G, K31H, K311, K31L, K31K, K31M, K31F, K31P, K31S, K31T, K31W, K31Y, K31V;
F28A, F28R, F28N, F28D, F28E, F28Q, F28G, F28H, F281, F28L, F28K, F28M, F28F, F28P, F28S, F28T, F28W, F28Y, F28V;
Y41A, Y41R, Y41N, Y41D, Y41E, Y41Q, Y41G, Y41H, Y411, Y41L, Y41K, Y41M, Y41F, Y41P, Y41S, Y41T, Y41W, Y41Y, and Y41V.

In one embodiment, the ACE2 protein or fragment thereof comprises one or more mutations selected from the group consisting of:
T27A, T27R, T27N, T27D, T27E, T27Q, T27G, T27H, T271, T27L, T27K, T27M, T27F, T27P, T27S, T27W, T27Y, T27V;
Q42A, Q42R, Q42N, Q42D, Q42E, Q42H, Q421, Q42L, Q42K, Q42M, Q42F, Q42P, Q42S, Q42T, Q42W, Q42Y, Q42V;
H34A, H34R, H34N, H34D, H34E, H34Q, H34G, H341, H34L, H34M, H34F, H34P, H34S, H34T, H34W, H34Y, H34V;
K31A, K31R, K31N, K31Q, K31H, K311, K31K, K31M, K31F, K31W, K31Y, K31V;
F28W, F28Y;
Y41H, and Y41F.

In one embodiment, the ACE2 protein or fragment thereof comprises one or more mutations selected from the group consisting of: T27Y, K31Y, K31H, H34A, H34V, and combinations thereof. In one embodiment, the ACE2 protein or fragment thereof comprises a In one embodiment, the ACE2 protein or fragment thereof comprises a K31Y mutation. In one embodiment, the ACE2 protein or fragment thereof comprises a K31H mutation. In one embodiment, the ACE2 protein or fragment thereof comprises a H34V mutation.

Advantageously, the present inventors have established that these specific mutations resulted in mutants which exhibited the slowest dissociation rate against viral spike proteins, such as the RBD of SARS-CoV-1 and/or SARS-Cov-2.

In one embodiment, the ACE2 protein or fragment thereof comprises one of the following combination of mutations:
T27Y/K31H/H34A,
T27Y/K31Y/H34A,
T27Y/K31H/H34V, or
T27Y/K31Y/H34V.

Advantageously, recombinant/fusion polypeptides comprising these combinations of triple mutations had significantly improved binding affinity against spike proteins, such as the RBD of SARS-CoV-1 and/or SARS-Cov-2 compared to polypeptides with single mutations.

In one embodiment, the ACE2 protein or fragment thereof comprises the following combination of mutations: T27Y/K31H/H34A. Advantageously, recombinant/fusion polypeptides comprising the T27Y/K31H/H34A exhibited the best overall binding affinity against viral spike proteins, such as the RBD of SARS-CoV-1 and/or SARS-Cov-2. Surprisingly, such triple mutant polypeptides also had significantly improved neutralising potency across a wide range of SARS-CoV-2 variants (>30 variants) and SARS-CoV-1 compared to polypeptides having a single T27Y, K31H or H34A mutation. Furthermore, the triple mutant polypeptides demonstrated high binding affinity to at least 5 bat SARS-like coronavirus strains.

In one embodiment, the recombinant/fusion polypeptide is capable of binding to a virus, optionally the recombinant/fusion polypeptide is capable of binding blocking/interfering/inhibiting/hindering/disrupting the binding of a virus (such as SARS-CoV-2) binding to a cell entry receptor.

In various embodiments, the recombinant/fusion polypeptide has a potent binding to a virus, such as SARS-CoV-2. In various embodiments, the recombinant/fusion polypeptide has a potent binding to a protein of a virus, such as SARS-CoV-2 (such as spike protein). In various embodiments, the recombinant/fusion polypeptide has a potent blocking/interfering/inhibiting/hindering/disrupting action against the binding of a virus, such as SARS-CoV-2 binding to the cell entry receptor. In various embodiments, the associated EC₅₀ value of the recombinant/fusion polypeptide is from about 0.1 nM to about 360 nM, from about 0.5 nM to about 100 nM, from about 0.5 nM to about 50 nM, from about 0.5 nM to about 25 nM, from about 0.5 nM to about 10 nM, from about 0.5 nM to about 9 nM or from about 0.5 nM to about 8 nM. In some embodiments, the associated EC₅₀ value of the recombinant/fusion polypeptide is no more than about 100 nM, no more than about 90 nM, no more than about 80 nM, no more than about 70 nM, no more than about 60 nM, no more than about 50 nM, no more than about 40 nM, no more than about 30 nM, no more than about 20 nM, no more than about 10 nM, no more than about 9 nM, or no more than about 8 nM, or no more than about 7 nM, or no more than about 6 nM, or no more than about 5 nM, or no more than about 4 nM, or no more than about 3 nM, or no more than about 2 nM, or no more than about 1 nM.

In some embodiments, the recombinant/fusion polypeptide is capable of neutralising or mediating the neutralisation of a virus, such as SARS-CoV-2, entry into cells, optionally human cells, further optionally ACE2 expressing/presenting cells.

In various embodiments, the recombinant/fusion polypeptide has a potent neutralising action against cell entry of a virus, such as SARS-CoV-2. In various embodiments, the associated EC₅₀ value of the recombinant/fusion polypeptide is from about 1 ng/ml to about 17000 ng/ml, from about 5 ng/ml to about 1000 ng/ml, from about 5 ng/ml to about 100 ng/ml, from about 5 ng/ml to about 60 ng/ml, from about 5 ng/ml to about 30 ng/ml or from about 5 ng/ml to about 12 ng/ml. In some embodiments, the associated EC₅₀ value of the recombinant/fusion polypeptide is no more than about 500 ng/ml, no more than about 100 ng/ml, or no more than about 50 ng/ml, or no more than about 40 ng/ml, or no more than about 30 ng/ml, or no more than about 20 ng/ml, or no more than about 10 ng/ml.

Therefore, in one embodiment, there is provided a recombinant/fusion polypeptide that is capable of recognizing/interacting with/binding to severe acute respiratory syndrome 2 (SARS-CoV-2) and/or severe acute respiratory syndrome 1 (SARS-CoV-1)

In one embodiment, the ACE2 protein or fragment thereof comprises one or more mutations that abolishes the natural peptidase activity of ACE2. The benefit of including such mutations is a reduced risk of unwanted side effects caused by ACE2's enzymatic activity.

In one embodiment, the ACE2 protein or fragment thereof comprises a mutation at amino acid position 273 and/or 505, for example positions 273 and/or 505 as set forth in SEQ ID NO: 24. Advantageously, mutations at positions 273 and/or 505 are able to affect the natural peptidase of ACE2.

In one embodiment, the ACE2 protein or fragment thereof further comprises a R273Q and/or a H505L mutation, such as both R273Q and H505L.

In one embodiment, the recombinant/fusion protein or fragment thereof comprising a R273Q and/or H505L mutation comprises the sequence:

(SEQ ID NO: 22)
QSTIEEQAKTFLDKFNHEAEDLFYQSSLASWNYNTNITEENVQNMNNAG
DKWSAFLKEQSTLAQMYPLQEIQNLTVKLQLQALQQNGSSVLSEDKSKR
LNTILNTMSTIYSTGKVCNPDNPQECLLLEPGLNEIMANSLDYNERLWA
WESWRSEVGKQLRPLYEEYVVLKNEMARANHYEDYGDYWRGDYEVNGVD
GYDYSRGQLIEDVEHTFEEIKPLYEHLHAYVRAKLMNAYPSYISPIGCL
PAHLLGDMWGQFWTNLYSLTVPFGQKPNIDVTDAMVDQAWDAQRIFKEA
EKFFVSVGLPNMTQGFWENSMLTDPGNVQKAVCHPTAWDLGKGDFRILM
CTKVTMDDFLTAHHEMGHIQYDMAYAAQPFLLRNGANEGFHEAVGEIMS
LSAATPKHLKSIGLLSPDFQEDNETEINFLLKQALTIVGTLPFTYMLEK
WRWMVFKGEIPKDQWMKKWWEMKREIVGVVEPVPHDETYCDPASLFLVS
NDYSFIRYYTRTLYQFQFQEALCQAAKHEGPLHKCDISNSTEAGQKLFN
MLRLGKSEPWTLALENVVGAKNMNVRPLLNYFEPLFTWLKDQNKNSFVG
WSTDWSPYADQSIKVRISLKSALGDKAYEWNDNEMYLFRSSVAYAMRQY
FLKVKNQMILFGEEDVRVANLKPRISFNFFVTAPKNVSDIIPRTEVEKA
IRMSRSRINDAFRLNDNSLEFLGIQPTLGPPNQPPVSEPKSCDKTHTCP
PCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVF
SCSVMHEALHNHYTQKSLSLSPGK.

In one embodiment, the recombinant/fusion protein or fragment thereof comprises an amino acid sequence selected from the group consisting of SEQ ID NOs: 1 to 21 as shown in Table 1 below.

TABLE 1
Amino acid sequences of recombinant/fusion proteins

| SEQ | | |
|---|---|---|
| ID NO: | Name | Amino Acid Sequences |
| 1 | ACE2-YHA_Fc | QSTIEEQAKYFLDHFNAEAEDLFYQSSLASWNYNTNITEENV |
| | (T27Y/K31H/H34A) | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 2 | ACE2-YYA_Fc | QSTIEEQAKYFLDYFNAEAEDLFYQSSLASWNYNTNITEENV |
| | (T27Y/K31Y/H34A) | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 3 | ACE2-YHV_Fc | QSTIEEQAKYFLDHFNVEAEDLFYQSSLASWNYNTNITEENV |
| | (T27Y/K31H/H34V) | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 4 | ACE2-YYV_Fc | QSTIEEQAKYFLDYFNVEAEDLFYQSSLASWNYNTNITEENV |
| | (T27Y/K31Y/H34V) | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 5 | ACE2-H34A_Fc | QSTIEEQAKTFLDKFNAEAEDLFYQSSLASWNYNTNITEENV |
| | | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 6 | ACE2-H34V_Fc | QSTIEEQAKTFLDKFNVEAEDLFYQSSLASWNYNTNITEENV |
| | | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 7 | ACE2-H34P_Fc | QSTIEEQAKTFLDKFNPEAEDLFYQSSLASWNYNTNITEENV |
| | | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 8 | ACE2-T27Y_Fc | QSTIEEQAKYFLDKFNHEAEDLFYQSSLASWNYNTNITEENV |
| | | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 9 | ACE2-T271_Fc | QSTIEEQAKIFLDKFNHEAEDLFYQSSLASWNYNTNITEENV |
| | | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 10 | ACE2-T27M_Fc | QSTIEEQAKMFLDKFNHEAEDLFYQSSLASWNYNTNITEENV |
| | | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 11 | ACE2-T27F_Fc | QSTIEEQAKFFLDKFNHEAEDLFYQSSLASWNYNTNITEENV |
| | | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 12 | ACE2-K31W_Fc | QSTIEEQAKTFLDWFNHEAEDLFYQSSLASWNYNTNITEENV |
| | | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 13 | ACE2-Q42L_Fc | QSTIEEQAKTFLDKFNHEAEDLFYLSSLASWNYNTNITEENV |
| | | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 14 | ACE2-H34D_Fc | QSTIEEQAKTFLDKFNDEAEDLFYQSSLASWNYNTNITEENV |
| | | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 15 | ACE2-T27Q_Fc | QSTIEEQAKQFLDKFNHEAEDLFYQSSLASWNYNTNITEENV |
| | | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 16 | ACE2-H34E_Fc | QSTIEEQAKTFLDKFNEEAEDLFYQSSLASWNYNTNITEENV |
| | | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 17 | ACE2-H34G_Fc | QSTIEEQAKTFLDKFNGEAEDLFYQSSLASWNYNTNITEENV |
| | | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | WVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 18 | ACE2-K31H_Fc | QSTIEEQAKTFLDHFNHEAEDLFYQSSLASWNYNTNITEENV |
| | | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 19 | ACE2-K31Q_Fc | QSTIEEQAKTFLDQFNHEAEDLFYQSSLASWNYNTNITEENV |
| | | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 20 | ACE2-K31Y_Fc | QSTIEEQAKTFLDYFNHEAEDLFYQSSLASWNYNTNITEENV |
| | | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |
| 21 | ACE2-K31M_Fc | QSTIEEQAKTFLDMFNHEAEDLFYQSSLASWNYNTNITEENV |
| | | QNMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQLI |
| | | EDVEHTFEEIKPLYEHLHAYVRAKLMNAYPSYISPIGCLPAHL |
| | | LGDMWGQFWTNLYSLTVPFGQKPNIDVTDAMVDQAWDAQ |
| | | RIFKEAEKFFVSVGLPNMTQGFWENSMLTDPGNVQKAVCHP |
| | | TAWDLGKGDFRILMCTKVTMDDFLTAHHEMGHIQYDMAYAA |
| | | QPFLLRNGANEGFHEAVGEIMSLSAATPKHLKSIGLLSPDFQ |
| | | EDNETEINFLLKQALTIVGTLPFTYMLEKWRWMVFKGEIPKD |
| | | QWMKKWWEMKREIVGVVEPVPHDETYCDPASLFLVSNDYS |
| | | FIRYYTRTLYQFQFQEALCQAAKHEGPLHKCDISNSTEAGQK |
| | | LFNMLRLGKSEPWTLALENVVGAKNMNVRPLLNYFEPLFTW |
| | | LKDQNKNSFVGWSTDWSPYADQSIKVRISLKSALGDKAYEW |
| | | NDNEMYLFRSSVAYAMRQYFLKVKNQMILFGEEDVRVANLK |
| | | PRISFNFFVTAPKNVSDIIPRTEVEKAIRMSRSRINDAFRLNDN |
| | | SLEFLGIQPTLGPPNQPPVSEPKSCDKTHTCPPCPAPELLGG |
| | | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK |

underlined are human Fc sequences;
mutations are in bold;
mutations that abolish ACE2 enzyme activity (R273Q and H505L) in bold italic.

In one embodiment, the recombinant/fusion protein or fragment thereof comprises an amino acid sequence selected from the group consisting of SEQ ID NOs: 1 to 4, for example SEQ ID NO: 1. Hence, in one embodiment the recombinant/fusion protein or fragment thereof comprises an amino acid sequence as set forth in SEQ ID NO: 1. In one embodiment the recombinant/fusion protein or fragment thereof comprises an amino acid sequence as set forth in SEQ ID NO: 2. In one embodiment the recombinant/fusion protein or fragment thereof comprises an amino acid sequence as set forth in SEQ ID NO: 3. In one embodiment the recombinant/fusion protein or fragment thereof comprises an amino acid sequence as set forth in SEQ ID NO: 4.

In one aspect, there is provided a polynucleotide encoding a recombinant/fusion polypeptide as defined above.

In various embodiments, polypeptides and/or polynucleotides as described herein may include polypeptides and/or polynucleotides having similarity or identity with the sequences as described herein. For example, the polypeptides and/or polynucleotides as described herein may include polypeptides and/or polynucleotides having an identity of at least 70%, at least 75%, at least 80%, at least 85%, at least 90%, at least 91%, at least 92%, at least 93%, at least 94%, at least 95%, at least 96%, at least 97%, at least 98%, at least 99% or 100% as compared to the sequences as described herein. The percentage sequence identity can be determined using methods known in the art, for example using programs such as BLAST, NBLAST, XBLAST, and the like.

In one aspect, there is provided a vector comprising a polynucleotide as defined above.

In some embodiments, the vector is selected from the group consisting of a plasmid, a viral particle, a phage, a baculovirus, a yeast plasmid, a lipid based vehicle, a polymer microsphere, a liposome, and a cell based vehicle, a colloidal gold particle, lipopolysaccharide, polypeptide, polysaccharide, a viral vehicle, an adenovirus, a retrovirus, a lentivirus, an adeno-associated viruses, a herpesvirus, a vaccinia virus, a foamy virus, a cytomegalovirus, a Semliki forest virus, a poxvirus, a pseudorabies virus, an RNA virus vector, a DNA virus vector and a vector derived from a combination of a plasmid and a phage DNA, further optionally wherein said polynucleotide is operatively linked to an expression control sequence(s) to direct peptide synthesis, even further optionally wherein the vector comprises one or more selectable marker genes to provide a phenotypic trait for selection of transformed host cells.

In one aspect, there is provided a host cell transfected with or comprising a vector as defined above.

Any suitable host cell/vector system may be used for expression of the DNA sequences encoding the molecule of the present invention. Bacterial, for example E. coli, and other microbial systems may be used or eukaryotic, for example mammalian, host cell expression systems may also be used. Suitable mammalian host cells include CHO, myeloma or hybridoma cells.

In one embodiment, the host cell may comprise a bacterial cell, a yeast cell, an animal cell e.g. a mammalian cell and/or a plant cell. In various embodiments, the host cell may be a mammalian cell, such as but not limited to Chinese Hamster Ovaries (CHO) cells.

In one aspect, there is provided a composition comprising the recombinant/fusion polypeptide as defined above. In one embodiment, the composition comprises a therapeutic composition or a pharmaceutical composition. Thus, in one aspect, there is provided a pharmaceutical composition and/or a therapeutic composition comprising the recombinant/fusion polypeptide as defined above.

In one embodiment, the composition further comprises a pharmaceutically acceptable excipient, a buffer or carrier.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the patient. A thorough discussion of pharmaceutically acceptable carriers is available in Remington's Pharmaceutical Sciences (Mack Publishing Company, N.J. 1991).

In one aspect, there is provided a recombinant/fusion polypeptide, or a composition as defined above for use as a therapy or drug.

In one aspect, there is provided a recombinant/fusion polypeptide, or a composition as defined above for use for use in treating and/or preventing a viral infection in a subject.

In one aspect, there is provided a use of the recombinant/fusion polypeptide of or a composition as defined above in the manufacture of a medicament for treating and/or preventing a viral infection in a subject.

In one aspect, there is provided a method of treating and/or preventing a viral infection in a subject in need thereof, comprising administering the recombinant/fusion polypeptide or a composition as defined above to the subject.

In one aspect, there is provided a method of neutralizing a virus in a subject in need thereof, comprising administering the recombinant/fusion protein and/or composition as defined above, for example wherein the virus is a coronavirus such a SARS-CoV-1 and/or SARS-CoV-2.

In one embodiment, the recombinant/fusion polypeptide or composition is administered to the subject by mode of administrations known in the art, including but not limited to intramuscularly, subcutaneously, intravenously, intraarterially, intraarticularly, intraperitoneally, intranasally, parenterally, and the like.

In one embodiment, the subject is a mammal, such as a monkey, rabbit, mouse, rat, pig or dog. In one embodiment, the subject is a human.

In one embodiment, the viral infection is caused by a virus from the coronaviridae family, such as coronavirus, in particular severe acute respiratory syndrome 1 (SARS-CoV-1), severe acute respiratory syndrome 2 (SARS-CoV-2), or the like.

In one aspect, there is provided a protein, composition, therapy, produce or method as described herein.

### BRIEF DESCRIPTION OF FIGURES

FIG. 1A shows the structural modelling of ACE2 binding to SARS-CoV-1 (Model 1) and SARS-CoV-2 (Model 2) viruses.

FIG. 1B shows a flow chart for computational prediction of higher affinity binding polypeptides

FIG. 2 shows the kinetic measurements for crude ACE2 polypeptide variants against SARS-CoV-1 or SARS-CoV-2 RBD protein immobilized on the Octet Biolayer Interferometry (BLI) sensor Octet Bilayer Interferometry (BLI) sensor.

FIG. 3A shows the dissociation rates (k_{off}) of the ACE2 variants against the SARS-CoV-2 RBD (COVID-19)

FIG. 3B shows the dissociation rates (k_{off}) of the ACE2 variants against the SARS-CoV-1 RBD (SARS)

FIG. 4A shows affinity measurements of the ACE2 variants against the RBD of the SARS-CoV-1 and SARS-CoV-2 viruses.

FIG. 4B shows kinetic measurements of the ACE2 variants against the RBD of the SARS-CoV-1 and SARS-CoV-2 viruses.

FIG. 5 shows the EC₅₀ values (ng/ml) of the ACE2 variants against the SARS-CoV-2, its variants and SARS-CoV-1 by the pseudovirus neutralization assays. The values represent the average of two independent measurements.

FIG. 6 shows the binding affinity of the ACE2-YHA-Fc variant and wild-type ACE2 to the RBDs of bat SARS-like coronaviruses was measured by BLI assay. Both ACE2-YHA-Fc and WT ACE2-Fc have R273Q/H505L mutations. Immobilized ACE2-Fc binding to the bat RBDs was tested using a range of MBP-TEV-RBD concentrations from 200 nM to 3.125 nM (in 2-fold dilution).

### EXAMPLES

Example embodiments of the disclosure will be better understood and readily apparent to one of ordinary skill in the art from the following discussions and if applicable, in conjunction with the figures. It should be appreciated that other modifications may be made without deviating from the scope of the invention. Example embodiments are not necessarily mutually exclusive as some may be combined with one or more embodiments to form new exemplary embodiments. The example embodiments should not be construed as limiting the scope of the disclosure.

### Example 1—Development of Higher Affinity ACE2 Polypeptide Variants

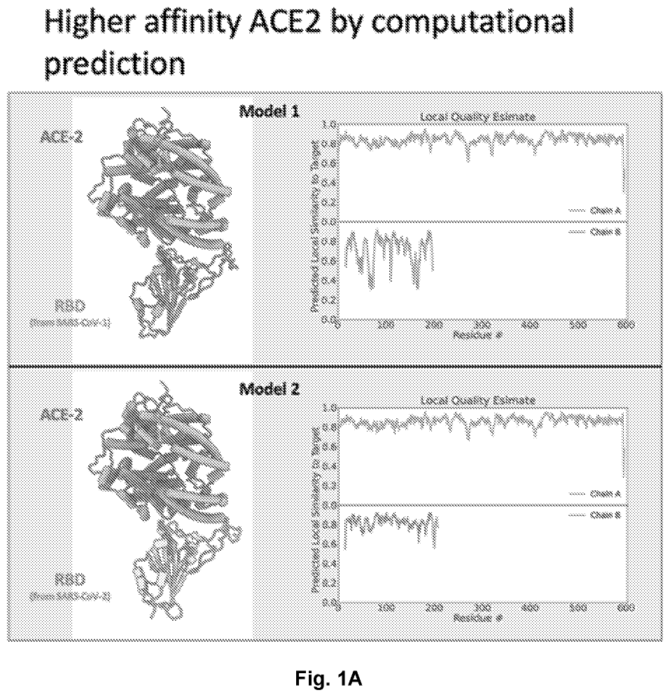

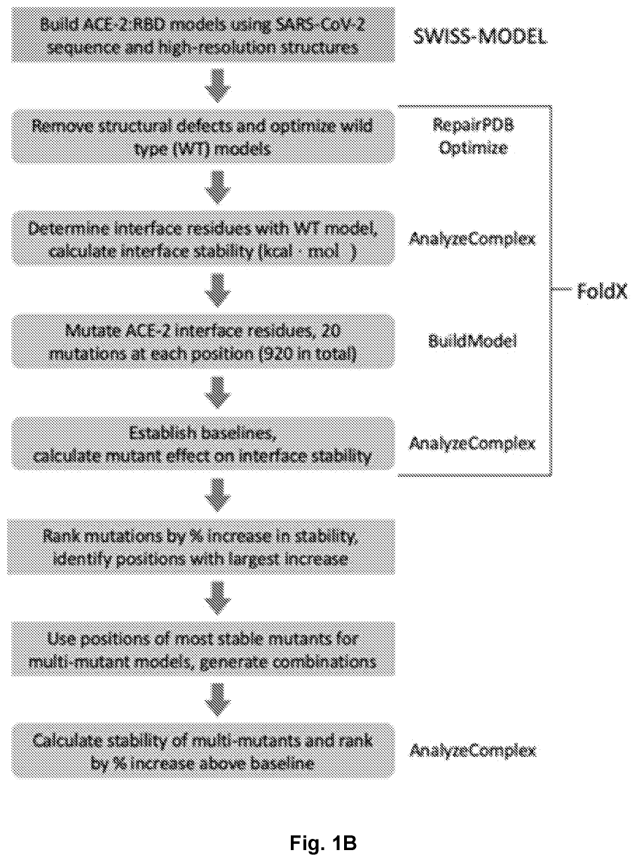

Affinity improvement engineering began with the structural modelling and computational prediction of amino acid positions which when mutated may lead to altered interaction with the viral spike proteins. FIG. 1A shows the structural modelling of ACE2 binding to SARS-CoV-1 (Model 1) and SARS-CoV-2 (Model 2) viruses. FIG. 1B shows a flow chart of the process for computational prediction of higher affinity binding ACE2 polypeptide variants.

ACE2 variants with mutations R273Q/H505L that abolish its natural peptidase activity were cloned as fusion proteins to human IgG1 Fc. Amino acids at positions 27, 28, 31, 34, 41 and 42 were selected by computational prediction for mutational studies. The amino acids at each position were replaced with all possible natural amino acids, except for the cysteine, by site-directed mutagenesis and the ACE2 mutants were expressed in ExpiCHO-S cells by transient transfection. Crude recombinant ACE2 mutants were then subjected to binding analysis by Bio-layer interferometry (BLI) to determine their dissociation from the receptor binding domain (RBD) of either SARS-CoV-1 or SARS-CoV-2.

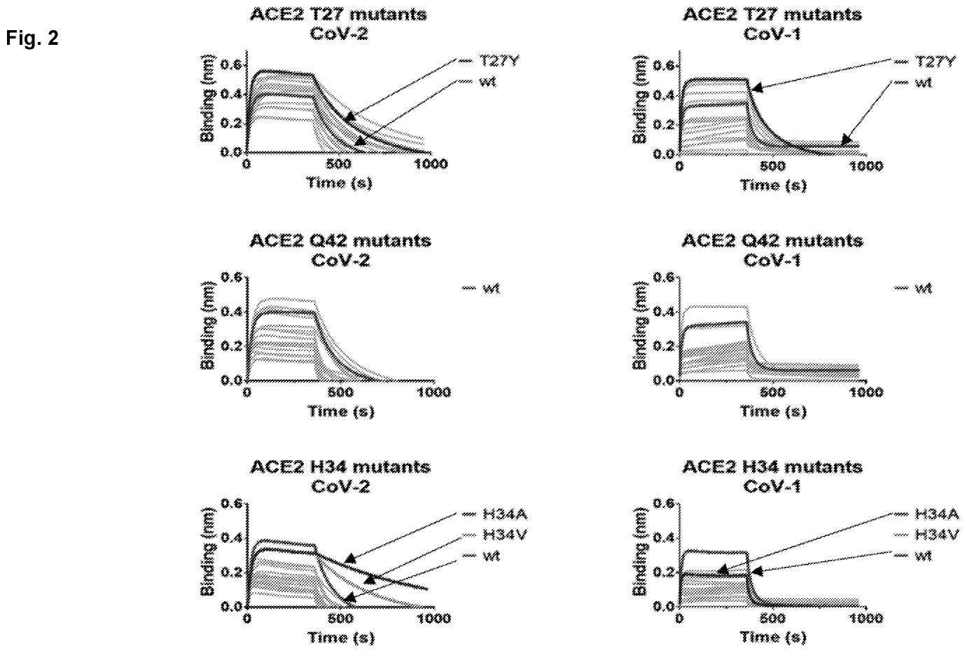

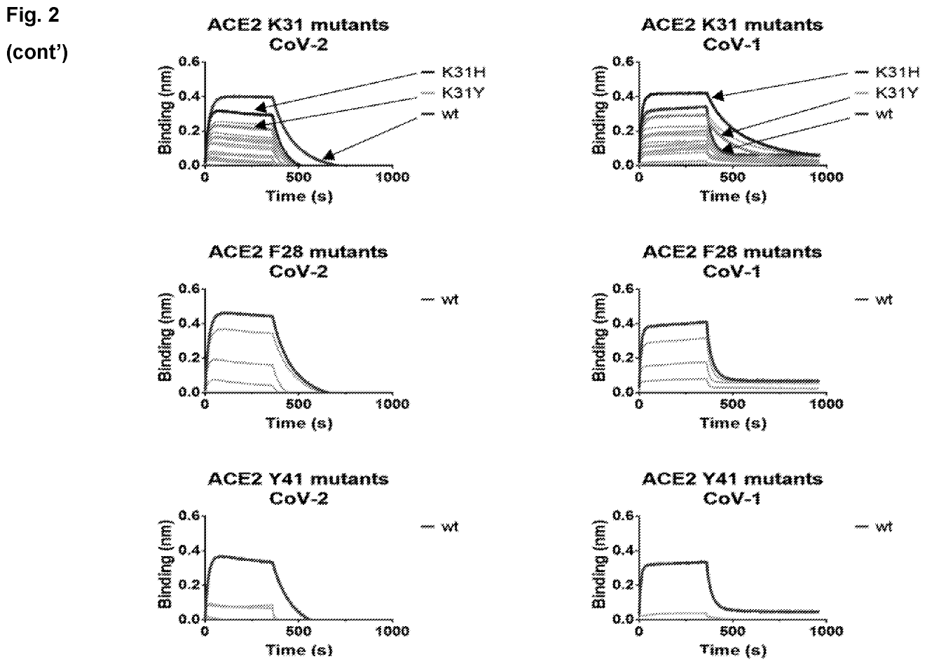

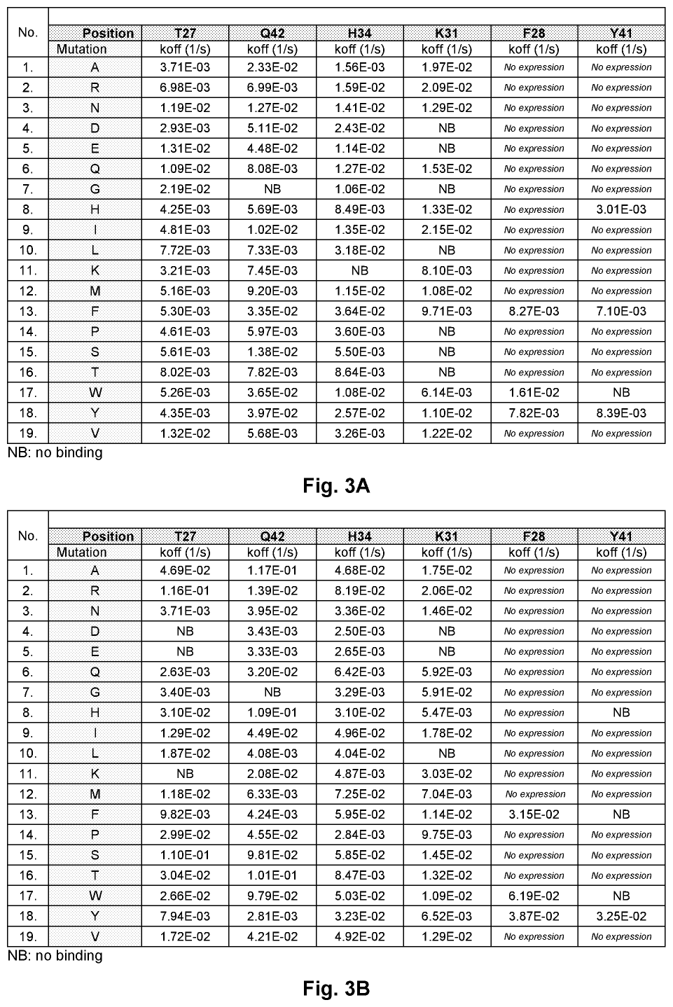

The results of the analysis are shown in FIG. 2 and FIGS. 3A & 3B. ACE2 variants that exhibited a slower dissociation rate against one or both antigens were selected and combined to form combinatorial mutants for further analysis.

### Example 2—Development of Combinatorial ACE2 Polypeptide Variants

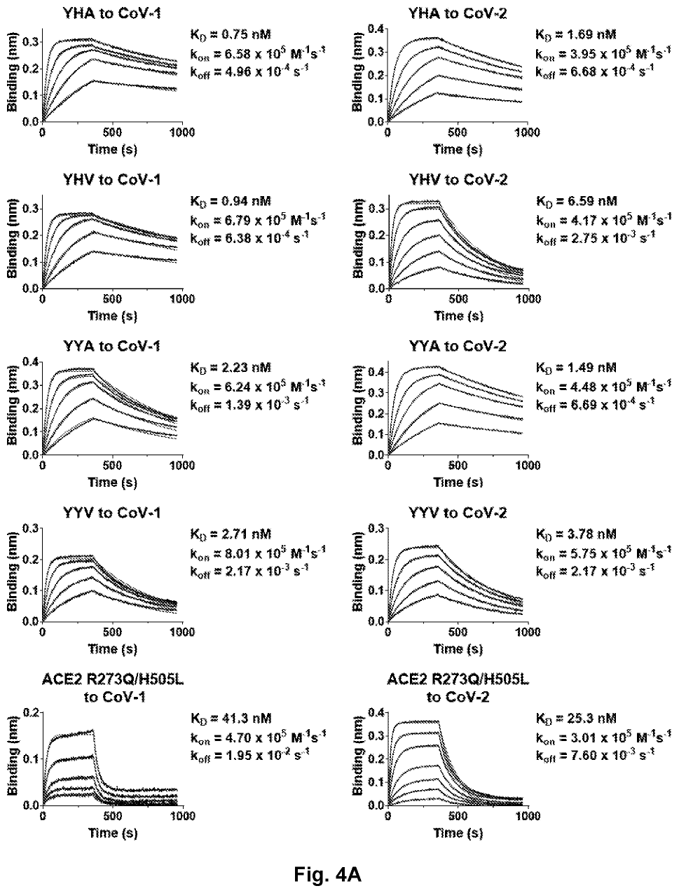

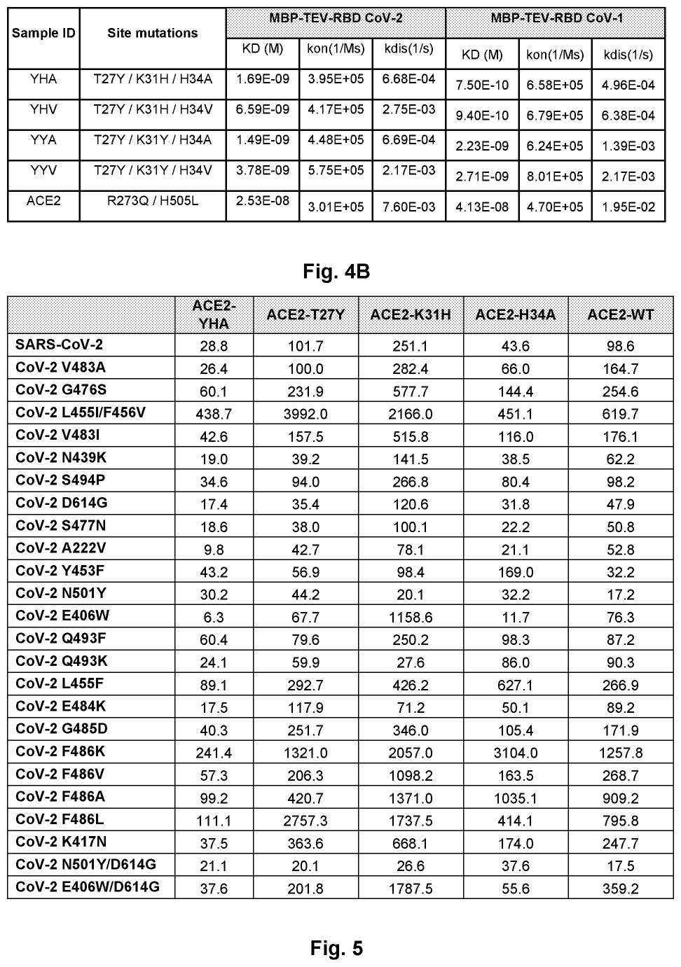

Four combinatorial ACE2 variants were produced: T27Y/K31H/H34A (YHA), T27Y/K31H/H34V (YHV), T27Y/K31Y/H34A (YYA), and T27Y/K31Y/H34V (YYV), all of which contain 2 additional mutation R273Q/H505L which abolish the enzymatic activity of ACE2. The affinity of these 4 ACE2 variants together with the wildtype ACE2 (also contains R273Q/H505L) were measured by the Biolayer Interferometry (BLI). The ACE2 proteins were captured on the Anti-Human IgG Fc (AHC) sensor and association with the Receptor Binding Domain (RBD) was measured in 2-fold dilutions starting from 200 nM to 3.125 nM. The RBD of the SARS-CoV-2 or SARS-CoV-1 used in the affinity measurement was fused to the C-terminus of the maltose binding protein (MBP). The results indicated that all triple-mutant combinations, T27Y/K31H/H34A (YHA), T27Y/K31H/H34V (YHV), T27Y/K31Y/H34A (YYA), and T27Y/K31Y/H34V (YYV), significantly improved the binding affinity against both SARS-CoV-1 and SARS-CoV-2 RBDs (see FIG. 4A). The optimal variant ACE2-YHA bound to SARS-CoV-1 RBD and SARS-CoV-2 RBD with KD values 0.75 nM and 1.69 nM, respectively, which is 55 and 15 times lower than the WT ACE2. The K31H mutation improved the binding affinity against SARS-CoV-1 RBD better than K31Y, and the H34A mutation enhanced SARS-CoV-2 RBD binding better than H34V. The affinity and kinetics results are shown in FIGS. 4A & 4B.

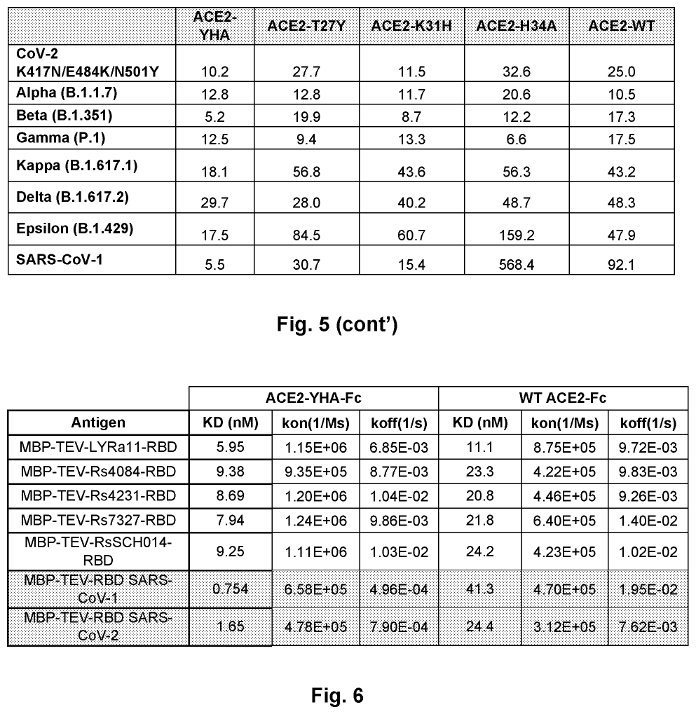

Subsequently, the neutralizing potency of the wildtype and mutant ACE2 against the SARS-CoV-1 and SARS-CoV-2 as well as a panel of the SARS-CoV-2 variants was measured by the pseudovirus neutralization assay. The EC₅₀ values are summarized in FIG. 5.

In general, the binding affinity was observed to correlate well with neutralization efficacy. Although the three single amino acid mutants (ACE2-T27Y, ACE2-K31H and ACE2-H34A) and the WT (wildtype) ACE2-Fc could neutralize all the pseudotyped viruses, they exhibited mixed efficacy in neutralizing different variants. In contrast, the ACE2-YHA retains high neutralizing potency across a wide range of SARS-CoV-2 variants and SARS-CoV-1, with IC₅₀ ranging from 5 ng/ml to 60 ng/ml in most cases. It exhibited significantly improved efficacy in SARS-CoV-1 and some SARS-CoV-2 variants such as E406W, E406W/D614G, and F486A, with more than 9-fold lower IC₅₀ than WT ACE2.

### Example 3—Binding Affinity of ACE2-YHA Variant Against Bat Coronaviruses

Next, the binding affinity of the ACE2-YHA variant across five bat SARS-like coronavirus strains LYRa11, Rs4084, Rs4231, Rs7327, RsSCH014 was tested.

The RBD sequences of LYRa11 and Rs7327 were known to closely resemble that of SARS-CoV-1, while Rs4231, RsSHC014, and Rs4084 showed more genetic differences from SARS-CoV-1 in the RBD region. The pseudotyped viruses expressing the spikes of Rs7327, Rs4231 and RsSHC014 could be replicated in human ACE2 expressing cells. Thus, the RBDs of these coronaviruses were produced and fused to the C-terminal of MBP proteins for a kinetic binding assay. The results of the assay are shown in FIG. 6.

ACE2-YHA showed 1.9-fold to 2.7-fold improvement of binding affinity comparing to the WT ACE2. It has previously been shown that recombinant WT ACE2 blocked the cell entry of pseudotyped viruses of these five strains, suggesting that the ACE2-YHA variant with improved binding affinity should also have cross-CoV neutralizing effects.

### APPLICATIONS

The present inventors have developed novel recombinant/fusion polypeptides comprising mutated angiotensin converting enzyme 2 (ACE2) with enhanced affinity against the spike proteins of SARS-CoV-1 and SARS-CoV-2 coronaviruses. In particular, the present disclosure demonstrates that the recombinant polypeptide known as ACE-YHA2 has the ability to neutralise pseudotyped viruses expressing spike proteins from over 30 existing SARS-CoV-2 variant strains.

Accordingly, the presently disclosed polypeptides are able to enhance neutralizing potency against SARS-CoV-1, SARS-CoV-2 and SARS-CoV-2 variant pseudoviruses and hence have the potential to function as universal therapeutics against viruses, such as a SARS-CoV-1 and SARS-CoV-2.

1. A recombinant/fusion polypeptide comprising a mutated angiotensin converting enzyme 2 (ACE2) protein, or fragment thereof, and an immunoglobulin fragment.

2. The recombinant/fusion polypeptide of claim 1, wherein the recombinant/fusion polypeptide comprises an immunoglobulin Fc fragment (region/domain), a protein capable of extending the half-life of the recombinant/fusion polypeptide (such as albumin, human albumin, and the like), linkers capable of enhancing binding valency (for example a rigid linker or a flexible linker, such as a GGGGS repeat), or combinations thereof, for example wherein the immunoglobulin Fc fragment is an IgG Fc fragment, such as a human IgG Fc fragment.

3. The recombinant/fusion polypeptide of claim 1, wherein the immunoglobulin fragment comprises the sequence:
(SEQ ID NO: 23)
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

4. The recombinant/fusion polypeptide of claim 1, wherein the recombinant/fusion polypeptide binds to a viral protein, or
wherein the viral protein is a Coronavirus protein or a Flaviviridae protein, or
wherein the coronavirus is a Severe Acute Respiratory Syndrome virus, such as severe acute respiratory syndrome 1 (SARS-CoV-1), severe acute respiratory syndrome 2 (SARS-CoV-2), and the like.

5. (canceled)

6. (canceled)

7. The recombinant/fusion polypeptide of claim 1, wherein the ACE2 protein or fragment thereof comprises one or more mutations, two or more mutations, three or more mutations, four or more mutations, five or more mutations, or six or more mutations, or seven or more mutations, or eight or more mutations, for example three mutations.

8. The recombinant/fusion polypeptide of claim 1, wherein the ACE2 protein or fragment thereof comprises one or more mutations at amino acid positions selected from the group comprising 27, 28, 31, 34, 41, and 42, for example positions 27, 28, 31, 34, 41 and 42 as set forth in SEQ ID NO: 24, and/or
wherein the ACE2 protein or fragment thereof comprises mutations at amino acid positions selected from the group comprising 27, 31 and 34, for example positions 27, 31 and 34 as set forth in SEQ ID NO: 24.

9. (canceled)

10. The recombinant/fusion polypeptide of claim 1, wherein the ACE2 protein or fragment thereof comprises one or more mutations selected from the group consisting of:
T27A, T27R, T27N, T27D, T27E, T27Q, T27G, T27H, T271, T27L, T27K, T27M, T27F, T27P, T27S, T27T, T27W, T27Y, T27V;
Q42A, Q42R, Q42N, Q42D, Q42E, Q42Q, Q42G, Q42H, Q42I, Q42L, Q42K, Q42M, Q42F, Q42P, Q42S, Q42T, Q42W, Q42Y, Q42V;
H34A, H34R, H34N, H34D, H34E, H34Q, H34G, H34H, H34I, H34L, H34K, H34M, H34F, H34P, H34S, H34T, H34W, H34Y, H34V;
K31A, K31R, K31N, K31D, K31E, K31Q, K31G, K31H, K31I, K31L, K31K, K31M, K31F, K31P, K31S, K31T, K31W, K31Y, K31V;
F28A, F28R, F28N, F28D, F28E, F28Q, F28G, F28H, F281, F28L, F28K, F28M, F28F, F28P, F28S, F28T, F28W, F28Y, F28V;
Y41A, Y41R, Y41N, Y41D, Y41E, Y41Q, Y41G, Y41H, Y41I, Y41L, Y41K, Y41M, Y41F, Y41P, Y41S, Y41T, Y41W, Y41Y, and Y41V.

11. The recombinant/fusion polypeptide of claim 1, wherein the ACE2 protein or fragment thereof comprises one or more mutations selected from the group consisting of:
T27A, T27R, T27N, T27D, T27E, T27Q, T27G, T27H, T27I, T27L, T27K, T27M, T27F, T27P, T27S, T27W, T27Y, T27V;
Q42A, Q42R, Q42N, Q42D, Q42E, Q42H, Q42I, Q42L, Q42K, Q42M, Q42F, Q42P, Q42S, Q42T, Q42W, Q42Y, Q42V;
H34A, H34R, H34N, H34D, H34E, H34Q, H34G, H34I, H34L, H34M, H34F, H34P, H34S, H34T, H34W, H34Y, H34V;
K31A, K31R, K31N, K31Q, K31H, K31I, K31K, K31M, K31F, K31W, K31Y, K31V;
F28W, F28Y;
Y41H, and Y41F.

12. The recombinant/fusion polypeptide of claim 1, wherein the ACE2 protein or fragment thereof comprises one or more mutations selected from the group consisting of: T27Y, K31Y, K31H, H34A, H34V, and combinations thereof.

13. The recombinant/fusion polypeptide of claim 1, wherein the ACE2 protein or fragment thereof comprises one of the following combination of mutations:
T27Y/K31H/H34A,
T27Y/K31Y/H34A,
T27Y/K31H/H34V, or
T27Y/K31Y/H34V.

14. The recombinant/fusion polypeptide of claim 1, wherein the ACE2 protein or fragment thereof comprises the following combination of mutations: T27Y/K31H/H34A.

15. The recombinant/fusion polypeptide of claim 1, wherein the recombinant/fusion polypeptide is capable of binding to a virus, optionally the recombinant/fusion polypeptide is capable of binding blocking/interfering/inhibiting/hindering/disrupting the binding of a virus (such as SARS-CoV-2) binding to a cell entry receptor, optionally
wherein the recombinant/fusion polypeptide is capable of neutralising or mediating (or initiating) the neutralisation of a virus, such as SARS-CoV-2, optionally
wherein the ACE2 protein or fragment thereof comprises one or more mutations that abolishes the natural peptidase activity of ACE2.

16. (canceled)

17. (canceled)

18. The recombinant/fusion polypeptide of claim 1, wherein the ACE2 protein or fragment thereof comprises a mutation at amino acid position 273 and/or 505, for example positions 273 and/or 505 as set forth in SEQ ID NO: 24.

19. The recombinant/fusion polypeptide of claim 1, wherein the ACE2 protein or fragment thereof further comprises a R273Q and/or a H505L mutation, such as both R273Q and H505L.

20. The recombinant/fusion polypeptide of claim 19, wherein the recombinant/fusion protein or fragment thereof comprising a R273Q and/or H505L mutation comprises the sequence:
(SEQ ID NO: 22)
QSTIEEQAKTFLDKFNHEAEDLFYQSSLASWNYNTNITEENVQNMNNAG
DKWSAFLKEQSTLAQMYPLQEIQNLTVKLQLQALQQNGSSVLSEDKSKR
LNTILNTMSTIYSTGKVCNPDNPQECLLLEPGLNEIMANSLDYNERLWA
WESWRSEVGKQLRPLYEEYVVLKNEMARANHYEDYGDYWRGDYEVNGVD
GYDYSRGQLIEDVEHTFEEIKPLYEHLHAYVRAKLMNAYPSYISPIGCL
PAHLLGDMWGQFWTNLYSLTVPFGQKPNIDVTDAMVDQAWDAQRIFKEA
EKFFVSVGLPNMTQGFWENSMLTDPGNVQKAVCHPTAWDLGKGDFRILM
CTKVTMDDFLTAHHEMGHIQYDMAYAAQPFLLRNGANEGFHEAVGEIMS
LSAATPKHLKSIGLLSPDFQEDNETEINFLLKQALTIVGTLPFTYMLEK
WRWMVFKGEIPKDQWMKKWWEMKREIVGVVEPVPHDETYCDPASLFLVS
NDYSFIRYYTRTLYQFQFQEALCQAAKHEGPLHKCDISNSTEAGQKLFN
MLRLGKSEPWTLALENVVGAKNMNVRPLLNYFEPLFTWLKDQNKNSFVG
WSTDWSPYADQSIKVRISLKSALGDKAYEWNDNEMYLFRSSVAYAMRQY
FLKVKNQMILFGEEDVRVANLKPRISFNFFVTAPKNVSDIIPRTEVEKA
IRMSRSRINDAFRLNDNSLEFLGIQPTLGPPNQPPVSEPKSCDKTHTCP
PCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVF
SCSVMHEALHNHYTQKSLSLSPGK.

21. The recombinant/fusion polypeptide of claim 1, wherein the recombinant/fusion protein or fragment thereof comprises an amino acid sequence selected from the group consisting of SEQ ID NOs: 1 to 21.

22. The recombinant/fusion polypeptide of a claim 1, wherein the 4 recombinant/fusion protein or fragment thereof comprises an amino acid sequence selected from the group consisting of SEQ ID NOs: 1 to 4, for example SEQ ID NO: 1.

23. (canceled)

24. (canceled)

25. (canceled)

26. A composition comprising:
a) the recombinant/fusion polypeptide of claim 1;
b) a polynucleotide encoding the recombinant/fusion polypeptide;
c) a vector comprising polynucleotide encoding the recombinant/fusion polypeptide; or
d) a host cell transfected with or comprising the vector comprising polynucleotide encoding the recombinant/fusion polypeptide.

27. (canceled)

28. (canceled)

29. (canceled)

30. A method of treating and/or preventing a viral infection in a subject in need thereof, comprising administering the recombinant/fusion polypeptide of claim 1 to the subject.

31. The method of claim 30, wherein the viral infection is caused by a virus from the coronaviridae family, such as coronavirus, in particular severe acute respiratory syndrome 1 (SARS-CoV-1), severe acute respiratory syndrome 2 (SARS-CoV-2), or the like.
